(19) United States
(12) Patent Application Publication
(10) Pub. No.: US 2020/0297243 A1
(43) Pub. Date: Sep. 24, 2020

(54) SYSTEM FOR ESTIMATING BODY MOTION OF PERSON OR THE LIKE

(71) Applicant: Toyota Jidosha Kabushiki Kaisha, Toyota-shi Aichi-ken (JP)

(72) Inventors: Yasuo Katsuhara, Susono-shi (JP); Hirotaka Kaji, Hadano-shi (JP)

(73) Assignee: Toyota Jidosha Kabushiki Kaisha, Toyota-shi Aichi-ken (JP)

(21) Appl. No.: 16/806,382

(22) Filed: Mar. 2, 2020

(30) Foreign Application Priority Data
Mar. 20, 2019 (JP) 2019-053799

(51) Int. Cl. A61B 5/11; A61B 5/00

(52) U.S. Cl. CPC A61B 5/11; A61B 5/6823; A61B 5/742; A61B 2562/0219; A61B 5/7275; A61B 5/7292; A61B 5/7264; A61B 5/0024

(57) ABSTRACT

In a system, a motion estimating unit is configured to output supervising reference position values of the plurality of regions of the body of a person or the like at a correct answer reference time at which a time difference from a measurement time is the same as a time difference between the reference time and the estimation time based on learning sensor-measured values over the first time length before the measurement time of the learning sensor-measured values using the learning sensor-measured values measured while the person or the like is performing a predetermined motion by learning according to an algorithm of a machine learning model and the supervising reference position values of the plurality of regions of a person or the like at the time of measurement thereof.

### CROSS REFERENCE TO RELATED APPLICATION

This application claims priority to Japanese Patent Application No. 2019-053799 filed on Mar. 20, 2019, incorporated herein by reference in its entirety.

### BACKGROUND

### 1. Technical Field

The disclosure relates to a system that can predict or estimate a motion of a body of a person or an animal (hereinafter referred to as a “person or the like”) in the future, the present, or the past and more particularly to a system that predicts or estimates a motion of a person or the like based on a measured value such as an acceleration value which is measured in the trunk of a person or the like.

### 2. Description of Related Art

Various techniques for detecting or analyzing an exercise or a motion of a person or the like have been proposed for various purposes such as health care, safety management, and improvement in sports skills. For example, Japanese Patent Application Publication No. 2012-343 (JP 2012-343 A) discloses a walking analysis system in which measurement sensors for detecting an angular velocity or acceleration are attached on both sides of the hip joint, the knee joint, or the foot joint of the right leg or the left leg of a walker, a joint angle of the hip joint, the knee joint, or the foot joint of the walker is calculated based on measurement data which is output from the measurement sensors, and the calculated joint angle is used to evaluate a walking state of the walker. Japanese Patent Application Publication No. 2016-112108 (JP 2016-112108 A) discloses a system in which a sensor for acquiring data associated with a motion state of a human body during exercise is attached to an ankle of one leg of the human body, the human body is caused to perform a predetermined calibration motion, parameters for defining an attached state of the sensor, a posture when standing on the one leg, a length of a lower thigh of the one leg, and the like are calculated, a moving image for reproducing a motion state of the legs when the human body walks is generated based on the parameters and data acquired from the sensor when the human body walks, and the generated moving image is displayed. “Computational Foresight: Forecasting Human Body Motion in Real-time for Reducing Delays in Interactive System” by Yuki Horiuchi and two others, ISS 2017, P. 312-317, proposes a configuration which acquires three-dimensional position information of 25 joints of a person and three-dimensional position information of the centers of gravity thereof when the person is jumping at a rate of 30 frames per second by motion capture, performs machine learning in a neural network using the acquired three-dimensional position information of the 25 joints and the centers of gravity thereof, calculates three-dimensional position information of the 25 joints and the centers of gravity thereof after 0.5 seconds have elapsed from the three-dimensional position information of the 25 joints and the centers of gravity thereof corresponding to 10 frames using the neural network, and predicts a motion after 0.5 seconds have elapsed in a jumping motion of a human body.

### SUMMARY

When a present motion of a person or the like can be detected or analyzed as described in JP 2012-343 A and JP 2016-112108 A and a future motion of a person or the like can be predicted as described in “Computational Foresight: Forecasting Human Body Motion in Real-time for Reducing Delays in Interactive System” by Yuki Horiuchi and two others, ISS 2017, P. 312-317, such prediction information can be expected to be advantageously used for various applications as will be specifically described later. In this case, a motion of a person or the like which is predicted may not be limited to a specific motion such as walking or jumping but includes various daily exercises or motions. Data which is used for a person or the like to predict a motion of the person or the like may be data which can be measured regardless of a place in a state in which a body orientation or an exercise of the person or the like is affected as little as possible the person or the like moves as usual. In this regard, the inventors found that a motion of a person or the like can be estimated by a machine learning model using data which has been measured by an acceleration sensor or the like attached to the trunk of the person or the like, and particularly that a future motion of a person or the like after a time point at which measurement by the sensor has been performed can be predicted. This knowledge is used in the disclosure.

The disclosure provides a system that can predict or estimate a motion including various exercises of a person or the like based on data which is measured by a sensor attached to the trunk of the person or the like.

According to the disclosure, there is provided a system that estimates a motion of a person or the like, the system comprising: a sensor that is attached to the trunk of the person or the like and measures a value varying with a motion of the person or the like as sensor-measured values in a time series; and a motion estimating unit configured to estimate estimated position values indicating positions at an estimation time of a plurality of predetermined regions of the body of the person or the like as the motion of the person or the like which is estimated at the estimation time using the sensor-measured values which are measured in a time series by the sensor over a first time length before a reference time. The motion estimating unit is configured to learn in accordance with an algorithm of a machine learning model such that supervising reference position values of the plurality of predetermined regions of the body of the person or the like are output at a correct answer reference time at which a time difference from a learning sensor-measurement time in learning data is the same as a time difference of the estimation time from the reference time based on learning sensor-measured values over the first time length before the learning sensor-measurement time at which the learning sensor-measured values in the learning data have been measured using the learning sensor-measured values which are measured in a time series by the sensor while the person or the like is performing a predetermined motion and the supervising reference position values which are acquired when the learning sensor-measured values have been measured and indicate the positions of the plurality of predetermined regions of the body of the person or the like as the learning data, and to output the estimated position values of the plurality of predetermined regions of the body of the person or the like which are estimated at the estimation time based on the sensor-measured values which are measured in a time series by the sensor over the first time length before the reference time.

In this configuration, a “person or the like” may be a person or an animal (or may be a walking type robot) as described above. A “motion” of a person or the like means that positions of regions of the body of the person or the like change because the person or the like changes a posture in various forms or moves hands or feet, and includes various exercises. A region of the “trunk of a person or the like” to which a sensor is attached may be an arbitrary region of the trunk of which a direction or a position changes with a motion of a person or the like, such as the head, the neck, the chest, the abdomen, the waist, or the hips of the person or the like. A “value varying with a motion of a person or the like,” that is, a “sensor-measured value,” is typically an acceleration value and may also be another value which varies with a motion of the person or the like such as an angular acceleration value. A “sensor” is typically an acceleration sensor and may also be a sensor that measures another value which varies with a motion of a person or the like. When an acceleration value is employed as a “sensor-measured value,” the acceleration value may be an acceleration value in at least one axis direction and may suitably be acceleration values in three different axis directions (the three axis directions are selected such that one axis crosses a plane including two different axes; the three axis directions are not necessarily perpendicular to each other). In this case, the “acceleration sensor” may be of an arbitrary type as long as it can measure acceleration values in three axis directions, and typically, a three-axis acceleration sensor that can measure acceleration values in three axis directions using one device is advantageously, used but the acceleration sensor is not limited thereto (three sensors that can each measure an acceleration value along one axis may be attached in three different directions for use). Typically, the sensor may be attached to only one region of the trunk of a person or the like such that a sensor-measured value is measured in the region to which the sensor is attached.

In this configuration, the “reference time” is a time which may be appropriately set by a user or a setter of the system, and when a future motion of a person or the like is predicted in the system according to the disclosure, the reference time is typically a present time, but may be a time prior to the present time. The “first time length” is a length of a time range of a sensor-measured value which is referred to by the motion estimating unit for the purpose of estimation of a motion of a person or the like and is a time length which may be appropriately set by a user of the system. However, since performance of estimation of a motion of a person or the like is determined by the first time length as will be described later, a preferable time length may be determined by experiment or the like. The “estimation time” is a time at which a motion of a person or the like which is estimated by the system according to the disclosure is performed. When this estimation time is set to a time after the reference time, a motion of a person or the like which is estimated is a future motion. In this case, a future motion of a person or the like can be predicted by the system (that is, estimation of a motion of a person or the like is prediction of a future motion of the person or the like.). The estimation time may be set to be the same as the reference time or may be set to be prior to the reference time. The “estimation time” may be set to an arbitrary time difference before or after the reference time. Since the performance of estimation of a motion of a person or the like is affected by the length of the time difference of the estimation time from the reference time, a suitable time difference may be determined by experiment or the like. In the following description, the length of the time difference between the reference time and the estimation time is referred to as a “second time length.”

In this configuration, a motion of a person or the like is expressed by positions of a plurality of predetermined regions of the body of the person or the like. Here, the plurality of predetermined regions may be at least two arbitrary regions of the body of the person or the like. Specifically, the plurality of predetermined regions may include the head, the spine, the right shoulder, the left shoulder, and the waist of a person or the like, may include the right leg, the left leg, the right foot, and the left foot of a person or the like, or may include the right arm, the left arm, the right hand, and the left hand of a person or the like. The plurality of predetermined regions is suitably selected such that the outline of the whole body of a person or the like can be ascertained (it should be understood that the number of predetermined regions of the body of a person or the like is greater than the number of regions which are measured by a sensor (typically, one)). The “estimated position values” are coordinate values of the positions of the plurality of predetermined regions, and typically, one region is expressed by coordinate values in three different axis directions. The estimated position values may further include coordinate values of the centers of gravity of the plurality of predetermined regions (an average value of the estimated position values in each axis direction).

In the above-mentioned configuration, the motion estimating unit is configured to output estimated position values of a plurality of predetermined regions of the body of a person or the like which is estimated at the estimation time based on sensor-measured values which are measured in a time series by the sensor over the first time length before the reference time by machine learning. A data set including learning sensor-measured values which are measured in a time series by the sensor while the person or the like is performing a predetermined motion and supervising reference position values indicating positions of the plurality of predetermined regions of the body of the person or the like which are acquired when the learning sensor-measured values have been measured is used as learning data which is used for the machine learning (the supervising reference position values may additionally include coordinate values of the center of gravity of the body of the person or the like (an average value of the reference position values of the plurality of predetermined regions in each axis direction)). Here, the “predetermined motion” includes various motions which may be arbitrarily set by a user, a designer, or a creator of the system, and may be suitably set to include motions of a person or the like which are predicted to be estimated by the system or motions close thereto. The “learning sensor-measured value” is a value which is measured similarly to the sensor-measured values while a person or the like is performing a predetermined motion, and the “supervising reference position values” are coordinate values indicating the actual positions of the plurality of predetermined regions of the body of the person or the like when the “learning sensor-measured values” have been acquired and are coordinate values in the same coordinate space as the “estimated position values.”

In machine learning in the system according to the disclosure, as described above, the learning sensor-measured values over the first time length before the learning sensor-measurement time at which the learning sensor-measured values have been measured, that is, a group of learning sensor-measured values in a section with the first time length, are used as input data and the “supervising reference position values” of the plurality of predetermined regions of the body of the person or the like at the “correct answer reference time” are used as correct answer data to perform learning. Here, the “supervising reference position values” are coordinate values of the positions of the plurality of predetermined regions similarly to the estimated position values and one region is typically expressed by coordinate values in three different axis directions. The “correct answer reference time” is selected such that a time difference from the learning sensor-measurement time is the same as a time difference between the reference time and the estimation time. Accordingly, when the estimation time is a time after the second time length has elapsed from the reference time, a time at which the second time length has elapsed from the learning sensor-measurement time (the final measurement time of the group of learning sensor-measured values which is used as the input data) is selected as the correct answer reference time. When the estimation time is a time before the second time length has elapsed from the reference time, a time before the second time length has elapsed from the learning sensor-measurement time is selected as the correct answer reference time (when the estimation time is equal to the reference time, the correct answer reference time is also equal to the learning sensor-measurement time.).

With the configuration of the system according to the disclosure, as described above, simply speaking, the motion estimating unit is configured by machine learning using a data set including learning sensor-measured values which are measured in a time series by the sensor while a person or the like is performing a predetermined motion and supervising reference position values indicating positions of a plurality of predetermined regions of the body of the person or the like which are acquired when the learning sensor-measured values have been measured as learning data, and a motion of the person or the like at the estimation time, that is, positions of a plurality of predetermined regions of the body of the person or the like, are estimated from the sensor-measured values which are measured over the first time length before the reference time by the sensor which is attached to the trunk of the person or the like using the motion estimating unit. Since regions of the body of a person or the like are connected to each other and positions of the regions of the body normally change continuously with the elapse of time in a motion of the person or the like (as long as it is not an extremely unnatural motion), a motion of a person or the like at a certain time point has a correlation with a state which is measured in an arbitrary region of the person or the like before and/or after the time point, for example, a part of the trunk thereof. Particularly, a sign of a motion of a person or the like at a certain time point appears in a state which is measured in the person or the like at a time point before the time point. A motion of a person or the like at a certain time point has an influence on states which are measured in the person or the like at the time point and a later time point.

Therefore, in the system according to the disclosure, a sign of a future motion of a person or the like (a motion which has not been performed yet) or an influence of a present or past motion of the person or the like is ascertained as a sensor-measured value by a sensor which is attached to the trunk of the person or the like and prediction of a future motion of the person or the like or estimation of a present or past motion of the person or the like is tried based on the sign of a future motion of the person or the like or the influence of a present or past motion of the person or the like in the sensor-measured values. In the system according to the disclosure, sensor-measured values acquired by the sensor which is attached to the trunk of the person or the like may be used as information which is used to estimate a motion of the person or the like. Such sensor-measured values can be measured in a state in which a direction or a motion of the body of the person or the like is affected as little as possible and the person or the like moves as usual. In this case, equipment which is not easily movable such as a motion capture system may not be used to acquire information which is used to predict or estimate a motion of the person or the like, and sensor-measured values can be collected at an arbitrary place as long as it is an environment in which the sensor which is attached to the person or the like can operate. Accordingly, a motion of a person or the like can be predicted or estimated regardless of a place in comparison with the related art. In the system, since the predetermined motion which is performed by a person or the like at the time of preparing learning data includes various motions, various daily exercises or motions can also be predicted or estimated. Accordingly, with the system according to the disclosure, it is possible to predict or estimate various motions of a person or the like from the past to the future based on data which is measured by a sensor which is attached to the trunk of the person or the like. As described above, the inventors of the disclosure experimentally ascertained through research and development that a motion of a person or the like after a time point at which sensor-measured values are acquired can be predicted based on the sensor-measured values which are acquired by a sensor such as an acceleration sensor attached to the trunk of the person or the like.

In the system according to the disclosure, since information which is used to predict or estimate a motion of a person or the like is sensor-measured values which are measured by a sensor attached to the trunk of the person or the like as described above, the same sensor-measured values are expected to be acquired with the same motion regardless of the direction in which the person or the like faces. That is, the sensor-measured values in the system are expressed as coordinate values in a coordinate space fixed to a person or the like. Accordingly, since the sensor-measured values in the system do not include information of a bearing or a direction which a person or the like faces and it is difficult to predict a bearing or a direction which the person or the like faces, estimated position values of a plurality of regions of the body of the person or the like indicating a motion of the person or the like which are estimated in the system may be expressed as coordinate values in the coordinate space fixed to the person or the like and supervising reference position values in learning data may be expressed similarly as coordinate values in the coordinate space fixed to the person or the like. The coordinate space fixed to the person or the like may be a coordinate space which is set such that a lateral direction of the person or the like is parallel to a predetermined direction, more specifically, a coordinate space which is set for the person or the like such that a lateral direction extending horizontally from the person or the like (for example, an extending direction of a projection of line connecting the right and left shoulders onto a horizontal plane) extends in a horizontal direction of the coordinate space and the vertical direction (the gravitational direction) extends in a vertical direction of the coordinate space.

In this regard, when information of positions of a plurality of predetermined regions of the body is acquired while a person or the like is performing a predetermined motion to acquire supervising reference position values which are used for correct answer data in learning data, the positions of the regions of the body of the person or the like are typically measured as coordinate values in a coordinate space fixed to an installation place of a system (referred to as a “position measurement space”) in the system that is installed in a certain place and includes a unit that measures positions of a plurality of predetermined regions of the body of the person or the like (a position measuring unit) such as a motion capture system. In this case, the measured coordinate values of the positions of the plurality of predetermined regions vary depending on a direction in which the person or the like faces or a position at which the person or the like is located in the position measurement space even when the person or the like performs the same motion. Then, when the machine learning is performed using the measured values of the positions of the regions of the body of the person or the like which are measured by the position measuring unit such as a motion capture system as correct answer data of the learning data, the coordinate values of the positions of the regions of the body of the person or the like vary depending on a direction in which the person or the like faces or a position at which the person or the like is located even when the person or the like performs the same motion (a plurality of different supervising reference position values may correspond to a learning sensor-measured value which is measured with the same motion) and thus correlation between the learning sensor-measured values and the supervising reference position values is excessively complicated and accurate learning may not be achieved. Accordingly, accuracy of a result of estimation of a motion of a person or the like in the system may deteriorate. This was found in experiments performed by the inventors of the disclosure as will be described later.

Therefore, in the system according to the disclosure, values obtained by measuring supervising measured position values which are coordinate values in a position measurement space indicating positions of a plurality of predetermined regions of the body of a person or the like while the person or the like is performing the predetermined motion using the position measuring unit that measures the coordinate values of the positions of the plurality of predetermined regions of the body of the person or the like in the position measurement space such as a motion capture system and performing a coordinate converting operation of converting the supervising measured position values from the position measurement space to the coordinate space fixed to the person or the like may be used as the supervising reference position values. With this configuration, since the supervising reference position values are expressed as coordinate values in the coordinate space fixed to the person or the like, it is possible to convert the coordinate values in the same motion into substantially the same values, to more simply correlate the supervising reference position values with the learning sensor-measured values in the learning data, to perform accurate learning, and to enhance performance of prediction of a motion of the person or the like using the system regardless of the direction in which the person or the like faces in the position measurement space or of the position at the time of measuring the positions of the regions of the person or the like. This was found through experiments performed by the inventors of the disclosure as will be described later.

In a coordinate converting operation of a system according to an embodiment, supervising measured position values of a pair of regions which have a symmetric positional relationship in a person or the like at each time point of learning data may be selected, a coordinate converting operation of matching a predetermined direction in the coordinate space fixed to the person or the like with an extending direction of a line connecting the selected supervising measured position values may be performed on the supervising measured position values of a plurality of predetermined regions of the body of the person or the like, and the supervising reference position values of the plurality of predetermined regions of the body of the person or the like may be calculated as coordinate values in the coordinate space fixed to the person or the like. Here, the “predetermined direction” in the coordinate space fixed to the person or the like may be arbitrarily set. In one embodiment, when the position measurement space is defined such that a z axis extends in the vertical direction and an x axis and a y axis extend in horizontal directions (the position measurement space is normally defined in this way), the coordinate converting operation corresponds to performing rotational coordinate conversion of supervising measured position values such that projection of the line connecting the supervising measured position values of a pair of regions which have a symmetric positional relationship of the person or the like onto the x-y plane in the position measurement space matches a predetermined direction by an angle between the projection of the line connecting the supervising measured position values of a pair of regions which have a symmetric positional relationship of the person or the like onto the x-y plane and a predetermined direction, for example, the x-axis direction (or it may be the y-axis direction). Accordingly, in this system, when the coordinate converting operation is performed, the rotational coordinate conversion may be performed on the supervising measured position values which are determined in the position measurement space and the supervising reference position values in the coordinate space fixed to the person or the like may be calculated (a specific operation process will be described later). At this time, a vertical axis (the z axis) of the coordinate space fixed to the person or the like is set to pass through a midpoint between a pair of regions which have a symmetric positional relationship of the person or the like, and the origin of the coordinate space fixed to the person or the like may be set to the midpoint (or, a projection of the midpoint onto the x-y plane is set as the origin of the coordinate space fixed to the person or the like).

As described above, in the system according to the disclosure, the motion estimating unit is configured by machine learning using learning data. In the machine learning, more specifically, parameters of a machine learning model which is set to calculate the supervising reference position values from the learning sensor-measured values using the learning data are typically determined in accordance with an algorithm of the machine learning model, and the estimated position values are output from the sensor-measured values in accordance with the algorithm of the machine learning model using the determined parameters at the time of estimating a motion of the person or the like. Accordingly, in a system according to an embodiment, a machine learning model parameter determining unit configured to determine parameters of a machine learning model in the motion estimating unit may be provided such that the motion estimating unit outputs supervising reference position values of a plurality of predetermined regions of a body of a person or the like at a correct answer reference time in the learning data based on learning sensor-measured values over the first time length before a learning data measurement time at which the learning sensor-measured values are measured in the learning data by the machine learning using the learning data, and the motion estimating unit may be configured to determine estimated position values using the determined parameters. As described above, since the supervising reference position values are values which are acquired by coordinate-converting the supervising measured position values obtained by a position measuring unit such as a motion capture system, a coordinate converting unit configured to acquire supervising reference position values by performing the coordinate converting operation of converting the supervising measured position values from the position measurement space to the coordinate space fixed to the person or the like may be provided in the system according to the embodiment.

In the system according to the disclosure, an arbitrary machine learning model that enables estimation of a motion of a person or the like at a time point after or before a time point at which sensor-measured values acquired by a sensor that is attached to the trunk of the person or the like such as an acceleration sensor are acquired based on the sensor-measured values may be selected as the machine learning model which is employed to construct the motion estimating unit. In one embodiment, the machine learning model may be a neural network. In this case, the motion estimating unit may be configured to output sensor-measured values which are measured over the first time length before the reference time or estimated position values of the regions of the person or the like at an estimation time at which features thereof are to be received as input data by machine learning of the neural network. Detailed settings of the neural network which is employed in the system according to the disclosure will be described later.

In the configuration of the system according to the disclosure, the sensor may be accommodated in a housing that can be attached to the trunk of a person or the like. A unit configured to perform an operation of estimating a motion of the person or the like from sensor-measured values may be accommodated in the housing or may be embodied by an external computer. In the system according to the disclosure, a motion at the estimation time which is output from the motion estimating unit may be displayed in an arbitrary display format, for example, in an image or a moving image. In this way, an estimated motion display unit configured to display the estimated position values of a plurality of predetermined regions of the body of the person or the like may be provided in the system according to the disclosure. A unit configured to output a result of estimation in the system according to the disclosure may be provided such that various other devices or systems operate based on the result of estimation in the system according to the disclosure.

In this way, with the system according to the disclosure, prediction or estimation of a motion of a person or the like can be achieved using sensor-measured values which are measured by the sensor which is attached to the trunk of the person or the like. In this configuration, since equipment for acquiring position information of regions of the body of the person or the like such as a motion capture system is used to collect learning data for learning of the motion estimating unit but prediction of a future motion of the person or the like or estimation of a past or present motion of the person or the like is possible using only the sensor-measured values as the input information after the learning has been performed, it is possible to use prediction information of a future motion of a person or the like or estimation information of a past or present motion of the person or the like without selecting a place when possible in a state in which a direction or an exercise of the body of the person or the like is affected as little as possible and the person or the like moves as usual.

Other objectives and advantages of the disclosure will become apparent from the following description of exemplary embodiments.

### BRIEF DESCRIPTION OF THE DRAWINGS

Features, advantages, and technical and industrial significance of exemplary embodiments will be described below with reference to the accompanying drawings, in which like numerals denote like elements, and wherein:

FIG. 1A is a diagram schematically illustrating a housing that is attached to the trunk of an examinee and includes a sensor for measuring a sensor-measured value (such as an acceleration value) and a computer terminal that estimates and displays a motion of the examinee in a motion estimation system according to an embodiment;

FIG. 1B is a block diagram illustrating an internal configuration of a motion estimation device in the motion estimation system according to the embodiment;

FIG. 1C is a block diagram illustrating a configuration of a machine learning device for machine learning of parameters of an estimator (a machine learning model) which is used for estimation of a motion in the motion estimation system according to the embodiment;

FIG. 2A is a diagram schematically illustrating an example of a motion of an examinee (a person or the like) changing from a standing position to a sitting position;

FIG. 2B is a diagram schematically illustrating an example of a motion of an examinee (a person or the like) changing from a standing position to a sitting position;

FIG. 2C is a diagram schematically illustrating an example in which a direction of an acceleration sensor changes in the motion of the examinee changing from a standing position to a sitting position;

FIG. 2D is a diagram illustrating a relationship between a time section of an acceleration value which is measured in a time series in the trunk of the examinee and a time period in which a state of the examinee is predicted, which is referred to for predicting a future motion of the examinee from the acceleration value;

FIG. 2E is a diagram illustrating a relationship between a time section of an acceleration value which is measured in a time series in the trunk of the examinee and a time period in which a state of the examinee is predicted which is referred to for estimating a present motion of the examinee from the acceleration value;

FIG. 2F is a diagram illustrating a relationship between a time section of an acceleration value which is measured in a time series in the trunk of the examinee and a time period in which a state of the examinee is predicted which is referred to for estimating a past motion of the examinee from the acceleration value;

FIG. 3A is a diagram illustrating a position measurement space when positions of a plurality of predetermined regions of the body of an examinee (a person or the like) are measured by a position measuring unit;

FIG. 3B is a diagram illustrating a coordinate space fixed to a person or the like (a fixed space of a person or the like);

FIG. 4A is a flowchart illustrating a process of estimating a motion of an examinee in the motion estimation system according to the embodiment;

FIG. 4B is a flowchart illustrating a process of determining parameters of an estimator (a machine learning model) which is used to estimate a motion according to the embodiment;

FIG. 4C is a diagram illustrating times at which a feature is calculated according to the embodiment;

FIG. 5 is a flowchart illustrating a process of estimating a motion of an examinee when different estimator parameters are used depending on a posture or a motion state of the examinee in the motion estimation system according to the embodiment;

FIG. 6A is a diagram illustrating examples (prediction) of a result of prediction of a body state of an examinee at an estimation time (after 0.5 seconds have elapsed from a reference time) which is estimated using an acceleration value before the reference time in the motion estimation system according to the embodiment, where the body state of the examinee (a correct answer) measured by a position measuring unit which corresponds to the result of prediction is illustrated for the purpose of comparison;

FIG. 6B is a diagram illustrating examples (prediction) of a result of prediction of a body state of an examinee at an estimation time (after 0.5 seconds have elapsed from a reference time) which is estimated using an acceleration value before the reference time in the motion estimation system according to the embodiment, where the body state of the examinee (a correct answer) measured by a position measuring unit which corresponds to the result of prediction is illustrated for the purpose of comparison;

FIG. 6C is a diagram illustrating examples (prediction) of a result of prediction of a body state of an examinee at an estimation time (after 0.5 seconds have elapsed from a reference time) which is estimated using an acceleration value before the reference time in the motion estimation system according to the embodiment, where the body state of the examinee (a correct answer) measured by a position measuring unit which corresponds to the result of prediction is illustrated for the purpose of comparison;

FIG. 6D is a diagram illustrating examples (prediction) of a result of prediction of a body state of an examinee at an estimation time (after 0.5 seconds have elapsed from a reference time) which is estimated using an acceleration value before the reference time in the motion estimation system according to the embodiment, where the body state of the examinee (a correct answer) measured by a position measuring unit which corresponds to the result of prediction is illustrated for the purpose of comparison;

FIG. 6E is a diagram illustrating examples (prediction) of a result of prediction of a body state of an examinee at an estimation time (after 0.5 seconds have elapsed from a reference time) which is estimated using an acceleration value before the reference time in the motion estimation system according to the embodiment, where the body state of the examinee (a correct answer) measured by a position measuring unit which corresponds to the result of prediction is illustrated for the purpose of comparison; and

FIG. 7 is a schematic diagram of a person or the like illustrating an application of the motion estimation system according to the embodiment.

### DETAILED DESCRIPTION

### Configuration of System

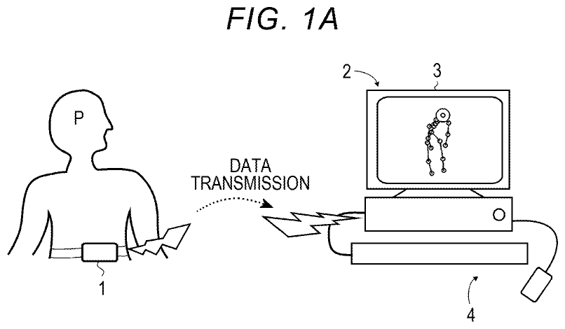

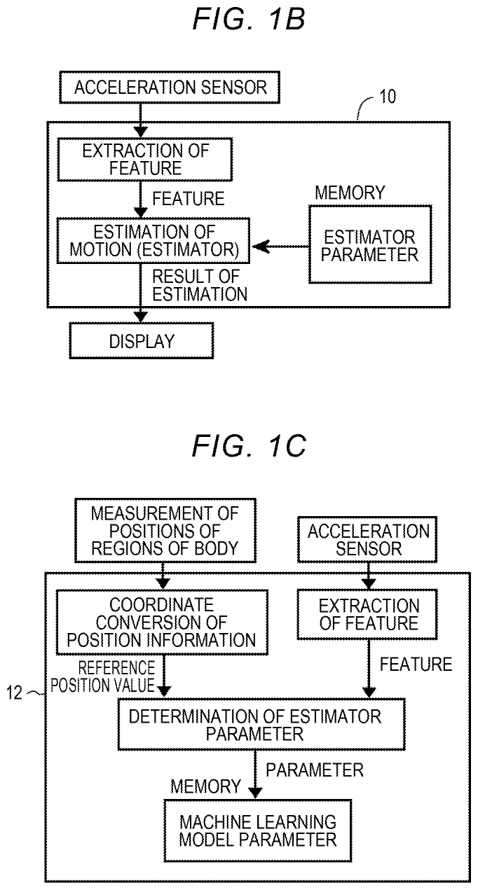

Referring to FIG. 1A, in a system for estimating a motion of a person or the like according to an embodiment, a housing 1 in which a sensor that measures a value which varies with a motion of an examinee P (a sensor-measured value) and which is referred to for estimation of the motion is accommodated is attached to the trunk of the examinee P who is a person or the like such as a head, a neck, a chest, an abdomen, a waist, and a hip. As illustrated in the drawing, the housing 1 may be formed with such a size by which a direction of a body or an exercise of an examinee P is affected as little as possible and in which the housing is relatively compactly portable such that measurement by the sensor is possible in a state in which the examinee is moving as usual. The sensor which is accommodated in the housing 1 is typically an acceleration sensor, and an acceleration value is employed as a sensor-measured value. In this case, an acceleration value may be an acceleration value in at least one axis direction or may be acceleration values in three axis directions which are different from each other. The three axis directions are selected such that one axis crosses a plane including two other axes. Typically, a three-axis acceleration sensor that can measure acceleration values in three axis directions with one device is advantageously used as the acceleration sensor, but the acceleration sensor is not limited thereto and may have an arbitrary format as long as it can measure acceleration values in three axis directions. An acceleration sensor with sensitivity that can measure a change of a projection distance of a gravity vector with respect to a measurement axis of an acceleration value due to a change in direction of the housing 1 following a change in direction of the trunk of an examinee P (that is, a change in component of the gravity vector in the acceleration measurement axis direction following a change in direction of the housing 1) and/or acceleration acting on the housing 1 with a motion of the examinee is used as the acceleration sensor. A gyro sensor or the like may be employed as the sensor, and another value varying with a motion of a person or the like such as an angular acceleration value may be employed as a sensor-measured value (The sensor-measured value may include a plurality of types of measured values). In the system according to the embodiment, typically, the number of sensors which are attached to the trunk of a person or the like may be one, and a sensor-measured value is measured in a region to which the sensor is attached (here, the number of sensors which are attached to the trunk of a person or the like may be two or more). It will be understood that the housing 1 in which the sensor is accommodated may have various shapes depending on the shape of a sensor which is employed.

The sensor-measured values which are measured by the sensor are transmitted in an arbitrary format to a motion estimating device 10 and estimation of a motion of an examinee P is performed in an aspect which will be described later. Typically, the motion estimating device 10 may be configured as a computer device 2 which is separate from the housing 1, and sensor-measured values which are outputs of the sensor may be transmitted to the computer device 2 by wired or wireless communication. In a general aspect, the computer device 2 includes a CPU, a storage device, and an input and output device (I/O) which are connected to each other via a bidirectional common bus, and the storage device includes a memory that stores programs for performing operation processes which are used for operation in the disclosure, and a work memory and a data memory which are used during operation. In a general aspect, the computer device 2 includes a monitor 3 and an input device 4 such as a keyboard and a mouse. When a program is started, a user can input various instructions to the computer device 2 using the input device 4 based on display on the monitor 3 in accordance with the program sequence and visually ascertain an estimation operation state, a result of estimation, and the like from the computer device 2 on the monitor 3. It should be understood that other peripheral devices (such as a printer which outputs a result and a storage device that is used to input and output calculation conditions, operation result information, and the like) which are not illustrated may be provided in the computer device 2.

The motion estimating device 10 may be provided in the housing 1, and a result of estimation may be output to the computer device 2 or other external device by an arbitrary format such as wired or wireless communication. In this case, a general small-sized computer device including a microcomputer for performing estimation of a motion and a memory or a flash memory may be accommodated in the housing 1. Some of processes of performing estimation of a motion of an examinee may be performed in the housing 1 and some thereof may be performed by an external computer device. A display that displays an output value of the motion estimating device 10 and/or an operation state of the device, a communication unit that transmits the output value to an external device or facility, an operation panel that receives an instruction and operation of the device by an examinee or a user, and the like may be provided in the housing 1.

In the motion estimating device 10, as illustrated in FIG. 1B, a feature extracting unit that normally receives sensor-measured values (acceleration values) which are measured by a sensor (an acceleration sensor) attached to the trunk of an examinee and extracts or calculates features from the sensor-measured values and a motion estimating unit that receives features from the feature extracting unit and estimates a motion of an examinee using an estimator which is configured using parameters (estimator parameters) which are stored in a memory with reference to the features are provided. A result of estimation of a motion is displayed on the display or is stored in an arbitrary storage device. It should be understood that operation of the units of the motion estimating device 10 are realized by execution of a program stored in the memory even when the motion estimating device 10 for the examinee is configured by the computer device 2 or is accommodated in the housing 1.

The estimator parameters which are used for a process of estimating a motion of an examinee are determined by machine learning in a machine learning device 12 and are stored in the memory as described above. The machine learning device 12 may be generally constituted by the computer device 2 or may be constituted by another computer and information of the estimator parameters may be transmitted to the computer device 2, but may be configured in the housing 1.

Referring to FIG. 1C, in the machine learning device 12, similar to the motion estimating device 10, a feature extracting unit that receives sensor-measured values (acceleration values) measured by a sensor (an acceleration sensor) and extracts or calculates features, a position information coordinate converting unit that receives measurement information of positions of a plurality of predetermined regions of a body of a person or the like which is measured using an arbitrary position measuring unit in parallel with measurement of the sensor-measured values by the sensor in a time series and performs a coordinate converting process of converting measured values of the positions of the regions of the body of the person or the like in an aspect which will be described later in detail, an estimator parameter determining unit that determines estimator parameters which are used to estimate a motion of an examinee in the motion estimating unit in accordance with an algorithm of a machine learning model using the features (input data) from the feature extracting unit and position information (correct answer data) of the regions of the body of the person or the like which has been subjected to coordinate conversion from the position information coordinate converting unit as will be described later in detail, and a memory that stores the estimator parameters may be provided.

Regarding the configuration of the machine learning device 12, a motion capture system is typically used as a position measuring unit that measures positions of a plurality of regions of the body of a person or the like to prepare supervised learning data, and the positions of the plurality of predetermined regions of the body of the person or the like are measured while the person or the like is performing various motions (predetermined motions) which may be arbitrarily determined. Specifically, the plurality of regions of the body of a person or the like may include regions which are arbitrarily selected such as the head, the neck, the spine, the right shoulder, the left shoulder, the waist, the right leg, the left leg, the right knee, the left knee, the right ankle, the left ankle, the right foot, the left foot, the right arm, the left arm, the right elbow, the left elbow, the right hand, and the left hand. Suitably, the plurality of predetermined regions may be selected such that the outline of the whole body of a person or the like can be ascertained. The positions of the regions of the body of the person or the like are typically measured in a time series as three-dimensional coordinate values in a coordinate space (a position measurement space) which is fixed to a place in which the motion capture system is installed or a place in which the person or the like who is to be measured is located. The coordinate values (measured position values) of the positions of the regions of the body of the person or the like can be measured, for example, using an image of the person or the like or based on outputs of an inertia sensor attached to each region of the body of the person or the like in an arbitrary aspect, and an arbitrary method may be selected in the system according to the embodiment as long as measured position values of the regions of the body of the person or the like can be acquired. The measured position values of the regions of the body of the person or the like may be typically acquired as coordinate values in a world coordinate system, but are not limited thereto.

In the configuration illustrated in FIG. 1C, the sensor (the acceleration sensor) and the feature extracting unit may be the same as those in the motion estimating device 10 (may be commonly used with the motion estimating device 10). It should be understood that operation of the units of the machine learning device 12 are realized by execution of a program stored in the memory even when the machine learning device 12 for the examinee is configured by the computer device 2 or is accommodated in the housing 1.

### Operation of Device (1)

### Principles and Summary of Motion Estimation

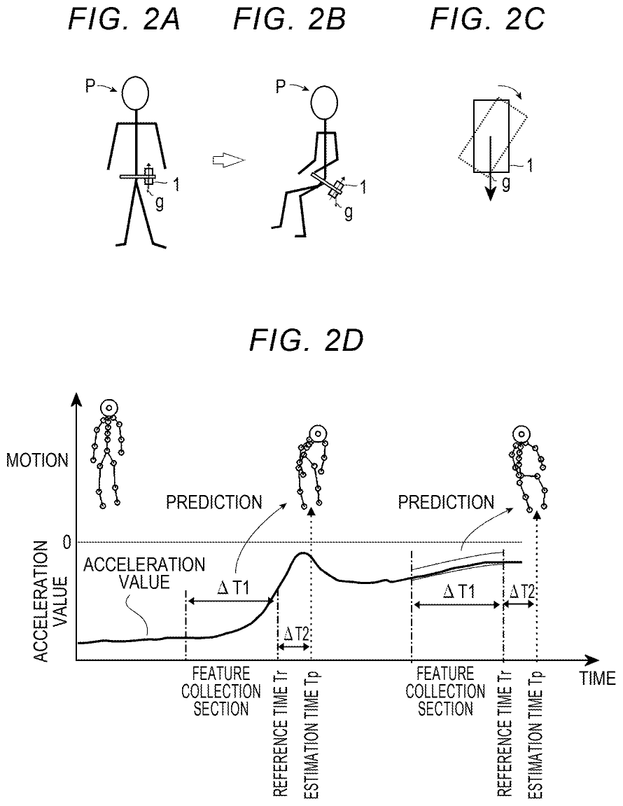

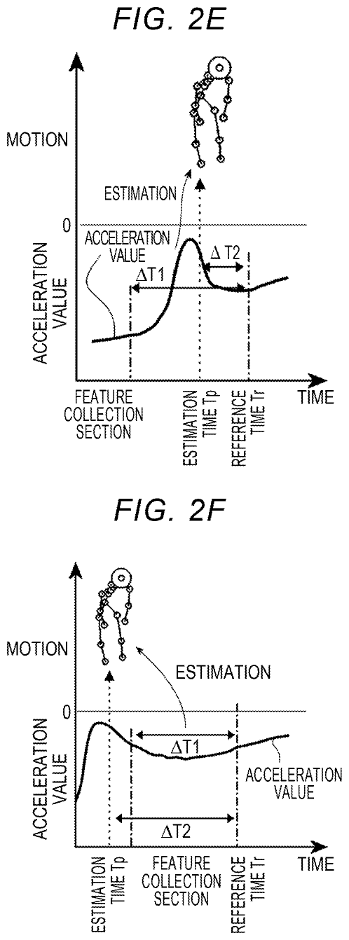

As described in the “SUMMARY,” since regions of the body of a person or the like are connected to each other, positions of the regions of the body of the person or the like have a correlation with a motion of a part of the trunk when the person or the like performs various motions. The motion of the trunk of the person or the like can be detected in a state in which it is measured by a sensor such as an acceleration sensor attached to the trunk of the person or the like (a value varying with a motion of the person or the like). For example, when an acceleration sensor is used, the direction of the acceleration sensor changes and the magnitude of an acceleration value corresponding to a gravity vector which is measured by the acceleration sensor 1 changes in a motion in which the posture of a person or the like P to which the acceleration sensor 1 is attached changes from a standing position (A) to a sitting position (B) as illustrated in FIGS. 2A to 2C, and thus the acceleration value which is measured by the acceleration sensor 1 with movement of the trunk is detected. When a person or the like performs various motions, positions of the regions of the body of the person or the like change continuously with the elapse of time as long as it is not an extremely unnatural motion, and thus the positions of the regions of the person or the like at a certain time point has a correlation with positions of the regions of the person or the like before and/or after the time point. Accordingly, since a sign of a motion of a person or the like at a certain time point is considered to appear in movement of the trunk of the person or the like at a time point before that time point, it is possible to predict a future motion of the person or the like by ascertaining a sign of a future motion of the person or the like (a sign indicating to what position each region moves) or a motion which is not performed yet in the motion of the trunk of the person or the like. That is, as schematically illustrated in FIG. 2D, a resultant state of a motion of a person or the like at an estimation time Tp, that is, positions of the regions of the body of the person or the like, can be predicted using a “sign of a motion” in movement of the trunk of the person or the like which is measured in a section of a time length ΔT1 before a reference time Tr which is a time length ΔT2 prior to the estimation time Tp. Similarly, since a motion of a person or the like at a certain time point is considered to be reflected in movement of the trunk of the person or the like at a time point before and/or after that time point (an influence of a motion appears), a present or past motion of the person or the like may be estimated by ascertaining an influence of the present or past motion of the person or the like in movement of the trunk of the person or the like. That is, as schematically illustrated in FIGS. 2E and 2F, a motion of a person or the like at an estimation time Tp, that is, positions of the regions of the body of the person or the like, can be estimated using an “influence of a motion” in movement of the trunk of the person or the like which is measured in a section of a time length ΔT1 before the reference time Tr which is a time length ΔT2 subsequent to the estimation time Tp.

Therefore, in the system according to the embodiment, simply speaking, an estimator (a motion estimating unit) that receives an input of sensor-measured values which are measured by a sensor attached to the trunk of a person or the like and predicts or estimates a motion of the person or the like (position information of regions of the body thereof) at a time point in the future, present, or past is constructed using an algorithm of a machine learning model, and prediction or estimation of a motion of the person or the like at a time point in the future, present, or past, that is, at an estimation time which is separated by an arbitrary time length ΔT2 (a second time length) from a reference time, from the sensor-measured values measured in a time series by the sensor attached to the trunk of the person or the like over a feature collection section ΔT1 (a first time length) before the reference time is performed using the estimator (see FIGS. 2D to 2F). In constructing the estimator, the estimator is constructed such that supervising reference position values of a plurality of predetermined regions of the body of the person or the like at a correct answer reference time (at a time at which a time difference from a learning data measurement time is the same as a time difference between the reference time and the estimation time) are output based on learning sensor-measured values over the feature collection section ΔT1 (a first time length) before a learning sensor-measurement time at which the learning sensor-measured values are measured through a learning process using the learning sensor-measured values measured in a time series by the sensor while the person or the like is performing various motions (predetermined motions) and the supervising reference position values indicating positions of a plurality of predetermined regions of the body of the person or the like which are acquired using the position measuring unit or the like when the learning sensor-measured values are measured as learning data in accordance with the algorithm of the machine learning model. An arbitrary model may be used as the machine learning model which is employed by the estimator as long as the above-mentioned input-output relationship can be acquired and, for example, a neural network is advantageously used as will be described later.

### (2) Coordinate Conversion of Position Information of Regions of Body of Person or the Like

In the system according to the embodiment, the sensor-measured values which are input to the estimator are values measured by the sensor which is attached to the trunk of an examinee P which is a person or the like and thus do not include information on a direction or a bearing of the examinee P (when the sensor-measured values are acceleration values, only the gravitational direction can be detected). That is, whatever direction or bearing the examinee P faces (in the gravitational direction), the sensor-measured values are normally the same when the same motion is performed. On the other hand, measured position values which are measured by the position measuring unit such as a motion capture system are normally used as position information of regions of the body of a person or the like which is used as learning data for machine learning of the estimator as described above. These measured position values are generally three-dimensional coordinate values in a position measurement space which is fixed to a place in which the position measuring unit is installed or a place in which a person or the like who is to be observed is located and thus are different from each other when directions with respect to the position measurement space are different even when the person or the like who is to be observed performs the same motion. When position information of the regions of the body of the person or the like which is expressed by the measured position values is used as learning data for the estimator without any change, the person or the like performs the same certain motion, and the sensor-measured values are the same, but when the direction in which the person or the like faces varies, the position information of the regions of the body of the person or the like varies and learning for the estimator may not be appropriately achieved (when there is a plurality of correct answer values for a certain input pattern, parameters of the estimator may not be uniquely determined and the output may be destabilized). Accordingly, in order to achieve appropriate learning, the position information of the regions of the body of the person or the like may be constant when the person or the like performs same motions. For this purpose, the position information of the regions of the body of the person or the like may be expressed by coordinate values in a coordinate space fixed to the person or the like (a fixed space of the person or the like) regardless of the direction of the person or the like.

In this way, in the system according to the embodiment, suitably, when position information of regions of the body of a person or the like for learning data for the estimator is expressed by coordinate values (measured position values) in the position measurement space of the position measuring unit, a process of converting the measured position values into coordinate values in the fixed space of the person or the like is performed and the coordinate values subjected to the coordinate conversion are used as correct answer data (supervising reference position values) in learning data.

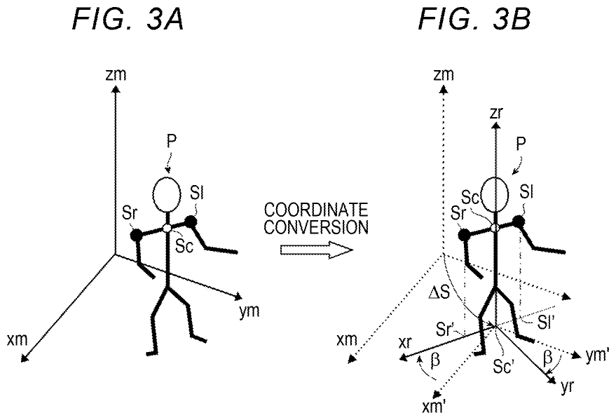

In an aspect of the coordinate conversion, when the coordinate values of the positions of the regions of the body of the person or the like are expressed by coordinate values in the position measurement space (xm-ym-zm) of the position measuring unit as illustrated in FIG. 3A, symmetric regions of the person or the like, for example, the right shoulder Sr and the left shoulder Sl, are selected (since what regions are the right shoulder Sr and the left shoulder Sl is known when the measured position values of the person or the like are measured, selection of such regions can be easily performed). Then, coordinate values of a midpoint between the two selected regions are calculated. That is, when the coordinate values of the right shoulder Sr and the left shoulder Sl are expressed as follows, respectively:

Sr=(Rx,Ry,Rz) (1a)

Sl=(Lx,Ly,Lz) (1b)

the coordinate values of the midpoint Sc are expressed as follows:

Sc=((Rx+Lx)/2,(Ry+Ly)/2,(Rz+Lz)/2) (2).

Here, the fixed space of the person or the like (xr-yr-zr) is defined with a projection point Sc′ of the midpoint Sc onto the xm-ym plane as an origin such that an extending direction of a projection line Sr′-Sl′ obtained by projecting a line Sr-Sl connecting the right shoulder Sr and the left shoulder Sl onto the xm-ym plane matches the xr axis. Then, coordinate conversion from the position measurement space to the fixed space of the person or the like corresponds to performing parallel movement of the positions of the regions of the body of the person or the like by Sc′ and rotation around the z axis by an angle β between the line Sr′-Sl′ and the xm axis. In this way, the measured position values A, expressed by the following equation (3), of the regions of the body of the person or the like first move in parallel by Sc′ and are converted into A⁺, expressed by the following equation (4).

A=(ax,ay,az) (3)

A⁺=A−Sc′ (4)

Here, Sc′ is expressed as follows:

Sc′=((Rx+Lx)/2,(Ry+Ly)/2,0) (5).

At this time, the right shoulder Sr is converted into Sr, expressed as follows:

Sr⁺=(Rx⁺,Ry⁺,Rz⁺)=Sr−Sc′ (6).

Then, the angle β between the line Sr′-Sl′ and the xm axis is given as the following equation β.

β=−arctan(Ry⁺/Rx⁺) (7).

Then, when A⁺ is rotated around the zm axis by the angle β as described below, converted coordinate values A⁺⁺ of the regions in which the positions of the regions of the body of the person or the like are expressed as coordinate values in the fixed space of the person or the like (the xr-yr-zr space) are obtained. The following (8) is satisfied.

A⁺⁺=C·A⁺ (8)

Here, C is expressed as follows:

$$C = ( \begin{matrix}cos β & - sin β & 0 \\ sin β & cos β & 0 \\ 0 & 0 & 1\end{matrix} ) \qquad ( 9 )$$

By performing coordinate conversion of the measured position values of the regions of the body of the person or the like which are acquired by the position measuring unit using Expressions (4) and (8), position information of the regions of the body of the person or the like which is used as supervising reference position values is expressed by coordinate values in the fixed space of the person or the like.

The coordinate converting method may be performed in arbitrary other manners. It is important that position information of regions of the body of a person or the like which is used as supervising reference position values is expressed by coordinate values in a space fixed to the person or the like. For example, the origin of the fixed space of the person or the like may be defined as the midpoint Sc. The right waist and the left waist or the like may be selected as the symmetric regions that determine the midpoint Sc. As long as the regions are expressed by the same coordinate values with the same motion of the person or the like, the z axis of the fixed space of the person or the like may not necessarily pass through the midpoint Sc of the person or the like. Any cases should be understood to belong to the scope of the disclosure.

### (3) Estimation of Motion

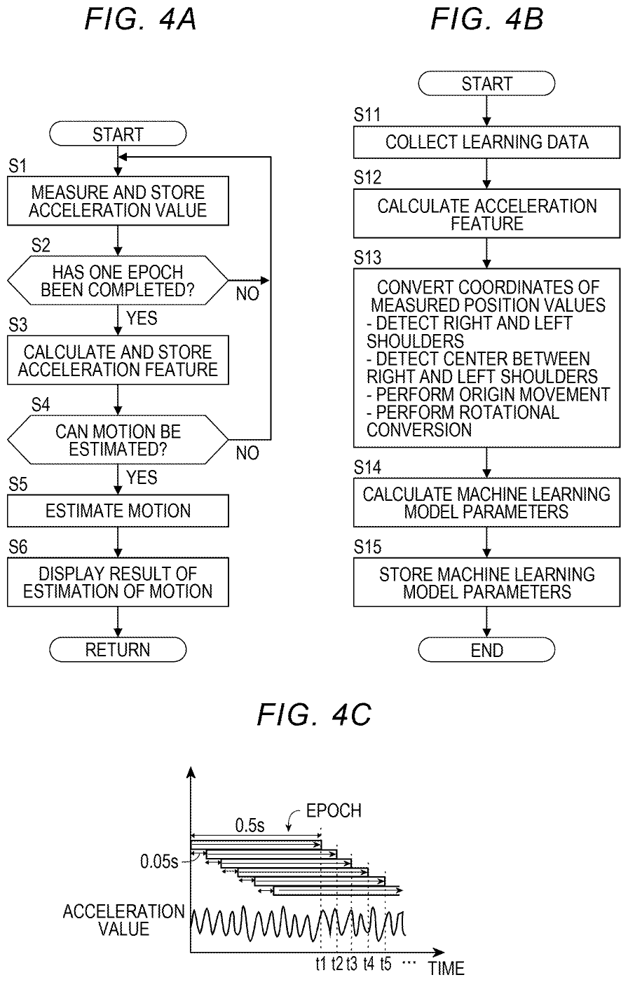

In estimating a motion of a person or the like in the system according to the disclosure, as illustrated in FIG. 4A, acquisition of sensor-measured values (acceleration values) (Step 1 to 2), calculation and storage of features (Step 3), estimation of a motion (Steps 4 to 5), display of a result of estimation (Step 6) may be sequentially performed and the result of estimation may be output. These processes will be described below.

(i) Acquisition of Sensor-Measured Values (Steps 1 to 3)

The sensor-measured values (acceleration values) which are measured for estimation of a motion are measured and stored by a sensor such as an acceleration sensor which is attached to the trunk of an examinee P as described above (Step 1). In the subsequent process, features which are calculated from time-series data of the measured values in the unit of an epoch as schematically illustrated in FIG. 4C may be used as input data of an estimator. In this case, the next process is performed whenever measurement of the sensor-measured values is completed for each epoch. One epoch may overlap a previous or subsequent epoch (a ratio of overlap with the previous or subsequent epoch may change arbitrarily) or may not overlap. As illustrated in the drawing, for example, an epoch with a length of 0.5 seconds may shift sequentially every 0.05 seconds, and features are extracted or calculated using time-series measured data in each epoch at the times Ct1, Ct2, . . . of end of each epoch. In this case, as illustrated in FIG. 4A, measurement and storage of the sensor-measured values (Step 1) are repeatedly performed until each epoch is completed, and the next process is performed whenever each epoch is completed (Step 2) (Step 3). The sensor-measured values for each predetermined time interval which may be arbitrarily set may be used as the features which are input to the estimator. In this case, measurement and storage of the sensor-measured values are not performed for each epoch, but measurement and storage of the sensor-measured values may be sequentially performed.

(ii) Calculation and Storage of Features (Step 3)

When the sensor-measured values are acquired, features of the sensor-measured values which are input to the estimator are extracted or calculated and stored. These features may be appropriately selected from time-series data in each epoch of the sensor-measured values. Typically, statistics for each predetermined time interval of the time-series data may be employed as the features. Specifically, for example, a maximum value, a minimum value, a median value, a variance, an autocorrelation value, or a periodogram (a frequency feature) of the sensor-measured values for each epoch can be used as the features. As described above, the features may be the sensor-measured values for each predetermined time interval itself. When acceleration values in three axis directions are used as the sensor-measured values, the features of the acceleration values in three axis directions are used. In this case, in each epoch or at each time point, three features are calculated or extracted. The features may be normalized (Z score conversion) after they have been calculated. A normalized feature X is given as follows:

X=(x−xₐ)/σₓ (10).

Here, x, xₐ, and σₓ represent a feature which is not normalized, an average value or a median value of all epochs thereof, and a standard deviation (xₐ and σₓ may be values in all epochs which have been measured hitherto). By this normalization, individual differences or intraindividual differences (differences due to conditions or seasons) can be removed and estimation accuracy can be improved.

(iii) Estimation of Motion (Steps 4 to 5)

When the features of the sensor-measured values are acquired in this way, whether estimation of a motion is possible is determined (Step 4). As described above, since the estimator that estimates a motion of an examinee in the system according to the embodiment is configured to output a motion state of an examinee P at an estimation time with reference to the features of the sensor-measured values over the feature collection section ΔT1 before the reference time, it may be determined that estimation of a motion is not possible and accumulation of features may be repeated without performing estimation of a motion until features over the feature collection section ΔT1 are accumulated (to Step 1). When the features over the feature collection section ΔT1 are accumulated (Step 4), a motion estimating process (Step 5) is performed.

In the motion estimating process (Step 5), as illustrated in FIGS. 2D to 2F, the estimator outputs estimated position values indicating coordinate values of positions of a plurality of predetermined regions of the body of an examinee P at the estimation time Tp as a result of estimation of a motion of the examinee P using a group of features in a time series over the feature collection section ΔT1 before the reference time Tr as input data. The estimated position values which are output may include an estimated position value of the center of gravity of the body of the examinee P (an average value of the estimated position values in the axis directions of the regions). Here, as described above, the estimator is constructed using parameters which are determined through a process based on an algorithm of a machine learning model in the machine learning device 12. An arbitrary model may be employed as the machine learning model as long as estimated position values of an examinee P at an estimation time Tp can be output from time-series features over the feature collection section ΔT1 before the reference time Tr by a learning process using sensor-measured values (learning sensor-measured values) which are measured in a time series by a sensor while a person or the like is performing a predetermined motion in advance and position information (supervising reference position values) indicating positions of a plurality of predetermined regions of the body of the person or the like acquired when the learning sensor-measured values are measured as learning data.

For example, when an estimator is constructed using a neural network as the machine learning model, features in the axis directions at time points in the feature collection section ΔT1 before the reference time Tr are given to neurons in an input layer of the neural network which is an input of the estimator, an operation using parameters determined in the machine learning device 12 is performed in an intermediate layer of the neural network, and estimated position values which are coordinate values in the axis directions of the positions of the plurality of predetermined regions of the body of the examinee P which are estimated at the estimation time Tp are output from the neurons in an output layer of the neural network which is an output of the estimator. The specific process of calculating estimated position values of the regions of the examinee P from the features may be performed based on an arbitrary algorithm, and may be typically performed using a function or a module which is prepared in a programming language. The positions of the regions which are specified by the estimated position values which are acquired herein indicate a body state of the examinee P which is estimated at the estimation time Tp and is a result of estimation of a motion in the system according to the embodiment. Particularly, in the system according to the embodiment, a motion of an examinee P when the estimation time Tp is subsequent to the reference time Tr can also be predicted. Regarding the estimated position values which are output from the estimator, when supervising reference position values supervising reference position values of regions of the body of a person or the like person or the like in learning data are expressed by coordinate values in a fixed space of the person or the like in the learning process of determining parameters of the estimator in the machine learning device 12, the estimated position values which are output from the estimator are also expressed by coordinate values in the fixed space of the person or the like.

(iv) Display of Result of Estimation (Step 6)

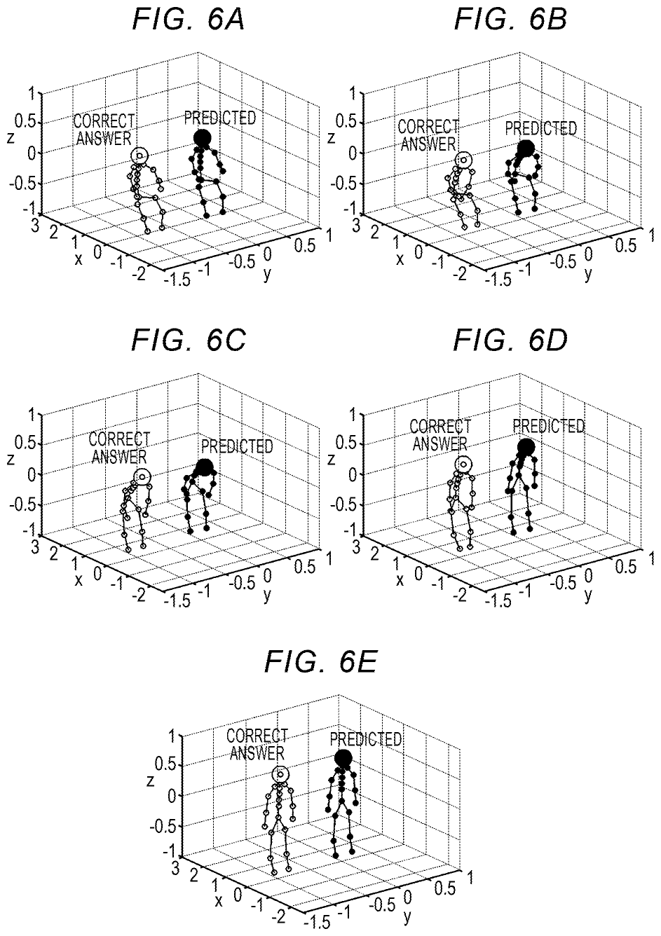

Positions of the regions of the body of the examinee P at the estimation time Tp can be estimated with reference to the estimated position values which are output in the motion estimating process. Therefore, the motion state of the body of the examinee P may be visualized, for example, as an image on the monitor 3 of the computer device 2 using the output estimated position values. In this case, plot points corresponding to the regions connected in the body may be connected by a line such that the motion of the examinee P can be easily recognized (see FIGS. 6A to 6E). An area in which the body of the examinee P is estimated to be located may be displayed. Alternatively, information of the estimated position values may be supplied to an external device or system such that they can be used for various applications.

The above-mentioned processes of a series have been described on the premise that estimation of a motion is performed in real time, but estimation of a motion over an arbitrary measurement period may be performed in a bundle after the sensor-measured values have been measured over the arbitrary period. In this case, features may be extracted from the acquired measured data and estimation of a motion may be performed using data of the extracted features (the determination process of Step 4 is not necessary).

### (3) Machine Learning for Estimator

As described above, in estimating a posture in the system according to the embodiment, parameters of an estimator for estimating a motion of an examinee are determined in advance by a machine learning method and are then stored in the memory.

Referring to FIG. 4B, in the machine learning process, first, measurement of sensor-measured values by a sensor attached to the trunk of a person or the like and measurement of positions of a plurality of predetermined regions of the body of the person or the like by the position measuring unit as described above are simultaneously performed in a time series to collect learning data while the person or the like is performing various motions (Step 11). Here, the person or the like who performs various motions for measurement of learning data may be the same person as an examinee who is subjected to estimation of a motion or may be another person. The person or the like who performs various motions for measurement of learning data and the person or the like who is an examinee of estimation of a motion may be close to each other in a type of constitution or a body height, but even when both persons or the like have a difference in a type of constitution or a body height, the difference can be interpolated by calculating similarity between the examinees using the body heights and the weights as interpolation information, whereby improvement in accuracy can be achieved. In a motion capture system, an influence of a difference in a type of constitution or a body height may be reduced by performing calibration of a motion using information of the body height or weight of the examinees. Various motions which are performed at the time of measurement of learning data can include motions which are daily performed by a person or the like such as a sitting motion, a standing motion, a walking motion, a prostrating motion, a stair ascending motion, a stair descending motion, and an object picking motion. The acquired sensor-measured values are learning sensor-measured values and are used to extract features which are used as input data in machine learning which will be described later. Here, position information of a plurality of predetermined regions of the body of a person or the like is used to prepare supervising reference position values supervising reference position values which are used as correct answer data in machine learning which will be described later.

When collection of learning data is completed, extraction or calculation of features from time-series data of the sensor-measured values and storage thereof are performed similarly to the case of Step 3 in FIG. 4A (Step 12). The types of the features are selected as the same types as used to estimate a motion in FIG. 4A. On the other hand, when the measured position values which are measured by the position measuring unit are coordinate values in the position measurement space fixed to a place in which a person or the like who is to be observed is located, a coordinate converting process of converting the measured position values into coordinate values in the fixed space of the person or the like is performed on the position information of a plurality of predetermined regions of the body of the person or the like which is measured by the position measuring unit as described above (Step 13). Specifically, for example, after right and left shoulders in the body of a person or the like are detected as expressed by Expressions (1a) and (1b) and the center between both shoulders is detected based on Expression (2), parallel movement expressed by Expression (4) and rotation expressed by Expression (8) may be performed on coordinate values A of each region of the body of the person or the like, and the converted coordinate values A⁺⁺ which are acquired as a result may be used as supervising reference position values which are referred to as correct answer data at the time of determining parameters of the estimator. When the measured position values measured by the position measuring unit are expressed by coordinate values in the fixed space of the person or the like, the measured position values may be used as the supervising reference position values without performing the coordinate converting process of Step 13 thereon.

By using the features of the sensor-measured values acquired in Step 12 as input data of an estimator and using the supervising reference position values acquired in Step 13 as correct answer data of the estimator, an operation of determining parameters of the estimator in accordance with an algorithm of a machine learning model is performed (Step 14—a learning operation). In this learning operation, when a group of features at time points in the feature collection section ΔT1 before a time point (a learning sensor-measurement time) at which the sensor-measured values which are used to calculate the features are measured is used as input data, the parameters of the estimator are determined such that the supervising reference position values of the regions of the body of the person or the like are output using supervising reference position values of the regions of the body of the person or the like at a correct answer reference time at which a time difference from the learning sensor-measurement time is the same as a time difference between the reference time Tr and the estimation time Tp as correct answer data (the supervising reference position values may include coordinate values of the center of gravity of the body of the person or the like (an average value of the supervising reference position values in the axis directions of the regions)). That is, as illustrated in FIG. 2D, in estimating a motion, when the estimation time Tp is a time point at which a time length ΔT2 has elapsed from the reference time Tr, the supervising reference position values at a time point at which the time length ΔT2 has elapsed from the learning sensor-measurement time are used as correct answer data. As illustrated in FIGS. 2E and 2F, in estimating a motion, when the estimation time Tp is a time point at which the time length ΔT2 has traced back from the reference time Tr, the supervising reference position values at a time point which is traced back by the time length ΔT2 from the learning sensor-measurement time are used as correct answer data. A specific operation process of determining the parameters of the estimator may be performed by an arbitrary algorithm and may be typically performed using a function or a module which is prepared in a programming language.

For example, when a neural network is used as the machine learning model as described above, parameters in each neuron (such as a weighting factor and a bias) may be calculated using an error back-propagation method or the like such that the supervising reference position values of the regions of the body of the person or the like (or supervising reference position values of the centers of gravity of the regions thereof in addition thereto) at the correct answer reference time are output to the neurons in an output layer of the neural network when features at time points in the feature collection section ΔT1 before the learning sensor-measurement time are input to neurons in an input layer of the neural network. [The learning operation may be performed at time intervals at which the supervising reference position values are acquired. Accordingly, the learning sensor-measurement time is set at the same time intervals as the time intervals at which the supervising reference position values are acquired. When features for each epoch are used as input data, the learning sensor-measurement time may be a final measurement time of the epoch.]

In this way, the estimator parameters which are determined by machine learning as described above are stored in the memory (Step 15) and are used to estimate a motion of the examinee.

In the system according to the embodiment, the number of regions which are measured and estimated in the body of an examinee P may be, for example, 21 (that is, the number of coordinate values of the regions of the body are about 63). As described above, the center of gravity of the body of a person or the like (the number of coordinate values is three) may be estimated. For example, the feature extraction time point (the reference time), the learning sensor-measurement time, and the correct answer reference time may be set with an interval of 0.05 seconds, and the feature collection section ΔT1 may be set with an interval of 0.5 seconds. The time difference between the reference time and the estimation time (that is, the time difference between the learning sensor-measurement time and the correct answer reference time) ΔT2 may be set to 0 to 2.0 seconds. In the learning process, for example, learning data including several thousands (for example, 3600) to several tens thousands of pieces of learning sensor-measured values and supervising reference position values may be used.

### (4) Improvement for Enhancement in Estimation Performance

In the system according to the embodiment, by learning sensor measured values which are measured when a person or the like performs various motions in advance, it is possible to estimate various motions of an examinee based on sensor-measured values which are measured from the examinee. In this regard, when a posture or a motion state of an examinee is narrowed to a certain extent using another method, estimation accuracy is expected to be improved by using an estimator which is constructed by learning using learning data acquired in the narrowed state.

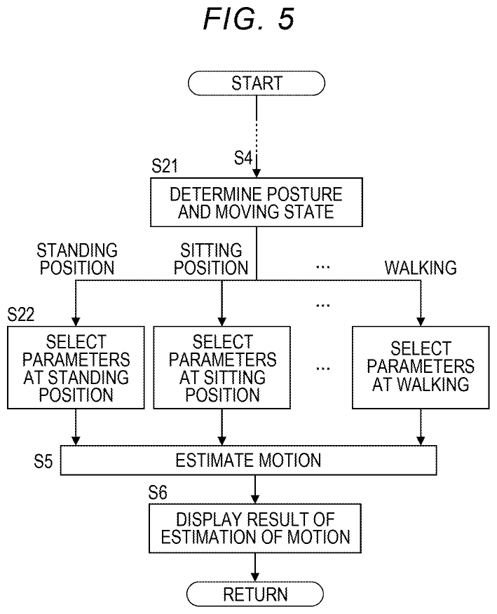

In this way, in another aspect of the system according to the embodiment, in estimating a motion, a posture or a motion state of an examinee is determined (Step 21) and a motion may be estimated using estimator parameters which are prepared for each determined state (Step 22) as illustrated in FIG. 5. Detection of a posture or a motion state of an examinee can be performed using an arbitrary method. The estimator parameters for each posture or each motion state can be calculated through the above-mentioned learning process using learning sensor-measured values which are measured while a person or the like is performing various motions with a posture or a motion state of the person or the like limited and position information of regions of the body of the person or the like.

### Test for Verification

The following test was performed to verify effectiveness of the embodiment described above. It should be understood that the following examples are for exemplifying effectiveness of the embodiment and does not limit the scope of the disclosure.

A test for estimating a motion of an examinee using sensor-measured values which are measured by a sensor attached to the examinee was performed with the system according to the embodiment. In the test, first, a housing in which a three-axis acceleration sensor is accommodated as a sensor was attached to a waist of an examinee, the examinee was caused to perform a motion (a test motion) of repeating a sitting motion and a standing motion three to four times per minute for three minutes in accordance with an instruction from a tester, acceleration values in three axis directions were measured using the three-axis acceleration sensor in the meantime, positions of the regions of the body of the examinee were measured by motion capture, and learning data and test data were collected. Measurement of the position of the regions of the body of the examinee was performed at 21 regions at intervals of 0.05 seconds (20 Hz). The examinee was caused to perform the test motion toward the north in collecting the learning data, another examinee was caused to perform the test motion toward the south in collecting the test data, and the positions of the regions of the bodies of the examinees were measured as coordinate values in the world coordinate system. The learning data was collected by causing three examinees to perform the test motion ten times (data corresponding to total 90 minutes was collected). The test data was collected by causing an examinee other than the examinees for the learning data to perform the test motion one time.

Thereafter, coordinate values which were obtained by a coordinate converting operation of converting the measured position values of the regions of the body of the examinee which are used for the learning data into a fixed space of a person or the like using Expressions (4) and (8) were used as supervising reference position values. Coordinate values of positions of the centers of gravity of 21 regions of the body of the examinee (an average value in each axis direction of the supervising reference position values of the 21 regions) were added to the supervising reference position values. Features of the sensor-measured values were set to values which were obtained by sampling the acceleration values in the three axis directions at intervals of 0.05 seconds. The length of the feature collection section ΔT1 was set to 1 second (corresponding to 20 groups of features every 0.05 seconds), and the estimation time Tp (the correct answer reference time) was set to a time point at which the time length ΔT2 of 0.5 seconds has elapsed from the reference time Tr (the learning sensor-measurement time).

In constructing an estimator, a neural network was used as a machine learning model. The configuration of the neural network was set to a four-layer configuration including an input layer, a hidden layer 1, a hidden layer 2, and an output layer. In the input layer, the number of neurons was set to 60, and values in the input data (acceleration values in the three axis directions×20 groups) were allocated to the neurons. In the hidden layer 1, the number of neurons was set to 4 and a sigmoid function was used as an activation function. In the hidden layer 2, the number of neurons was set to 66 and a sigmoid function was used as an activation function. In the output layer, the number of neurons was set to 66, a linear transfer function was used as an activation function, and supervising reference position values in the three axis directions of 21 regions of the body of an examinee and supervising reference position values in the three axis directions of the centers of gravity thereof were allocated to the neurons. A Levenberg-Marquardt method was used as a learning algorithm for determining parameters of an estimator (such as weighting factors and biases of the neurons). In learning, all the learning data which was collected by causing three examinees to perform the test motion 10 times was used.

The features of the sensor-measured values of the test data were input to the estimator using the parameters which were determined in accordance with the algorithm of the neural network in this way, and estimated position values of the regions of the body of each examinee were calculated. FIGS. 6A to 6E illustrate a motion state (prediction) of each examinee obtained by plotting the estimated position values of the regions of the body of the examinee acquired by the estimator and a motion state (a correct answer) of each examinee obtained by plotting the measured position values in the body of the examinee in the test data at the corresponding estimation time. In the drawings, a predicted image of each examinee is illustrated such the direction thereof matches a direction of an image of the examinee in the test data. As can be seen from FIGS. 6A to 6E, the motion state of each examinee predicted by the estimator of the system according to the embodiment matched substantially the actual motion state of the examinee. An average of differences of Euclidean distances between the positions of the regions of the body of the examinee in the test data (after coordinate conversion) and the positions of the regions of the body of the examinee predicted by the estimator (an average prediction error) was 10 cm. The average prediction error in only the trunk of the examinee was further decreased. It was also ascertained that estimation performance of the same degree as described above was obtained even when data obtained by causing each examinee to perform the test motion two times for 30 minutes was used as the learning data. It was ascertained that a motion could be estimated to the same extent even when learning data obtained by causing each examinee to perform a walking motion and an object gripping motion as the test motion. When the measured position values of the regions of the body of each examinee were used as the supervising reference position values without any change in learning, a motion in the test data was not estimated well. From these results, it was seen that details of a motion in a direction or a range of behavior in the future could be predicted or estimated with the system according to the embodiment.

With the system according to the embodiment, as long as there is an environment in which sensor-measured values can be measured by a sensor attached to an examinee, a future, present, or past motion of the examinee can be estimated in a state in which the examinee moves as usual regardless of a place, and thus the system can be used in various applications.

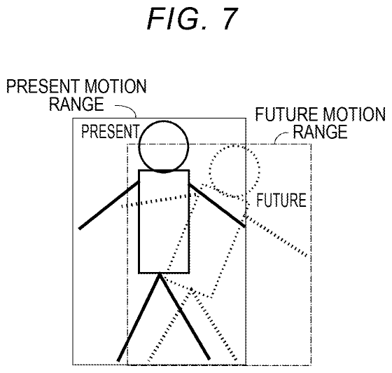

For example, with the system according to the embodiment, since a motion range in the future of an examinee can be predicted as schematically illustrated in FIG. 7, the system can be used to prevent contact (interference) with a surrounding object or person in a factory, a physical distribution center, or the like. Specifically, when a person or an object is located in a predicted motion range, the object or the person may be made to move or an alarm may be issued in order to prevent contact with the object or the person.

In the field of medical care, when a patient is an examinee of the system, a motion which is to be performed by the patient can be predicted and thus the system can be advantageously used to predict an accident or for a staff or a robot to understand various motions of the patient such as drinking water or turning over or to assist the motions at appropriate times.

In the field of sports, when a sportsman is an examinee of the system, a future motion of the sportsman can be predicted and thus the system can be considered to be used for skill enhancement such as habit curing training. For example, in training of predicting a future motion of a goalkeeper and stopping a kick at the time of a penalty kick in soccer training of reading a pick-off throw of a pitcher in baseball, a sportsman can use estimation of a motion in the system for training of recognizing and improving a habit of the sportsman in combination with AR technology.

With the system according to the embodiment, when learning is performed, estimation or prediction of a motion of a person or the like is possible using sensor-measured values from a sensor attached to the person or the like and thus the system is expected to be advantageously used for analysis of a motion of a person or the like in various fields.

While an embodiment of the disclosure has been described above, it will be apparent to those skilled in the art that the disclosure can be subjected to various corrections and modifications and the disclosure Is not limited to the embodiment but can be applied to various devices without departing from the concept of the disclosure.

What is claimed is:

1. A system that estimates a motion of a person or the like, the system comprising:
a sensor that is attached to a trunk of the person or the like and measures a value varying with a motion of the person or the like as sensor-measured values in a time series; and
a motion estimating unit configured to estimate estimated position values indicating positions at an estimation time of a plurality of predetermined regions of a body of the person or the like as the motion of the person or the like which is estimated at the estimation time using the sensor-measured values which are measured in a time series by the sensor over a first time length before a reference time,
wherein the motion estimating unit is configured to learn in accordance with an algorithm of a machine learning model such that supervising reference position values of the plurality of predetermined regions of the body of the person or the like are output at a correct answer reference time at which a time difference from a learning sensor-measurement time in learning data is the same as a time difference of the estimation time from the reference time based on learning sensor-measured values over the first time length before the learning sensor-measurement time at which the learning sensor-measured values in the learning data have been measured using the learning sensor-measured values which are measured in a time series by the sensor while the person or the like is performing a predetermined motion and the supervising reference position values which are acquired when the learning sensor-measured values have been measured and indicate the positions of the plurality of predetermined regions of the body of the person or the like as the learning data, and to output the estimated position values of the plurality of predetermined regions of the body of the person or the like which are estimated at the estimation time based on the sensor-measured values which are measured in a time series by the sensor over the first time length before the reference time.

2. The system according to claim 1, wherein the estimation time is a time after a second time length has elapsed from the reference time.

3. The system according to claim 1, wherein the sensor is an acceleration sensor and the sensor-measured values are acceleration values.

4. The system according to claim 3, wherein the sensor-measured values are acceleration values in three different axis directions.

5. The system according to claim 1, wherein the sensor is attached to only one region of the trunk of the person or the like and the sensor-measured values are measured in the region to which the sensor is attached.

6. The system according to claim 1, wherein the plurality of predetermined regions of the body of the person or the like include a head, a spine, a right shoulder, a left shoulder, and a waist of the person or the like.

7. The system according to claim 6, wherein the plurality of predetermined regions of the body of the person or the like further include a right leg, a left leg, a right foot, and a left foot of the person or the like.

8. The system according to claim 7, wherein the plurality of predetermined regions of the body of the person or the like further include a right arm, a left arm, a right hand, and a left hand of the person or the like.

9. The system according to claim 1, wherein the supervising reference position values and the estimated position values are expressed by coordinate values in a coordinate space which is fixed to the person or the like.

10. The system according to claim 9, wherein the coordinate space which is fixed to the person or the like is set such that a lateral direction of the person or the like is parallel to a predetermined direction.

11. The system according to claim 9, wherein the supervising reference position values are values obtained by measuring supervising measured position values which are coordinate values in a position measurement space indicating the positions of the plurality of predetermined regions of the body of the person or the like while the person or the like is performing a predetermined motion using a position measuring unit configured to measure the coordinate values of the positions of the plurality of predetermined regions of the body of the person or the like in the position measurement space and performing a coordinate converting operation of converting the supervising measured position values from the position measurement space to the coordinate space fixed to the person or the like.

12. The system according to claim 11, wherein the supervising reference position values of the plurality of predetermined regions of the body of the person or the like are calculated in the coordinate converting operation by selecting the supervising measured position values of a pair of regions with a symmetric positional relationship of the person or the like at each time point of the learning data and performing a coordinate converting operation of matching a predetermined direction in the coordinate space fixed to the person or the like with an extending direction of a line connecting the selected supervising measured position values on the supervising measured position values of the plurality of predetermined regions of the body of the person or the like.

13. The system according to claim 1, further comprising a machine learning model parameter determining unit configured to determine parameters of a machine learning model in the motion estimating unit such that the motion estimating unit outputs the supervising reference position values of the plurality of predetermined regions of the body of the person or the like at the correct answer reference time in the learning data based on the learning sensor-measured values over the first time length before the learning sensor-measurement times at which the learning sensor-measured values in the learning data are measured,
wherein the motion estimating unit is configured to determine the estimated position values using the parameters.

14. The system according to claim 1, wherein the machine learning model is a neural network, and
wherein the motion estimating unit is configured to output the estimated position values of the person or the like at the estimation time when the sensor-measured values measured over the first time length before the reference time or features thereof are received as input data by machine learning of the neural network.

15. The system according to claim 1, further comprising an estimated motion display unit configured to display the estimated position values of the plurality of predetermined regions of the body of the person or the like at the estimation time which are output from the motion estimating unit.
